(19) United States
(12) Patent Application Publication
(10) Pub. No.: US 2015/0041558 A1
(43) Pub. Date: Feb. 12, 2015

(54) NEBULIZER

(71) Applicants: Jens BESSELER, Bingen (DE); Holger HOLAKOVSKY, Witten (DE); Herbert ARGAUER, Pirk (DE); Josef GATZ, Moosbach (DE); Andreas GORSHOEFER, Nittenau (DE); Robert WALCH, Weiden (DE)

(72) Inventors: Jens BESSELER, Bingen (DE); Holger HOLAKOVSKY, Witten (DE); Herbert ARGAUER, Pirk (DE); Josef GATZ, Moosbach (DE); Andreas GORSHOEFER, Nittenau (DE); Robert WALCH, Weiden (DE)

(73) Assignee: Boehringer Ingelheim International GmbH, Ingelheim am Rhein (DE)

(21) Appl. No.: 14/453,806

(22) Filed: Aug. 7, 2014

(30) Foreign Application Priority Data
Aug. 9, 2013 (EP) 13003988.6

(51) Int. Cl. B05B 11/00; A61M 15/00; A61M 11/00

(52) U.S. Cl. CPC B05B 11/0008; A61M 11/006; A61M 15/009
USPC 239/302

(57) ABSTRACT

A nebulizer and a housing part for a nebulizer are proposed, wherein a container containing the fluid to be nebulized is provided. The container is moveable back and forth for conveying and nebulization of the fluid. A securing device forms a transportation lock for holding the container unmovable in a delivery state with closed transportation lock. A control part opens the transportation lock by means of at least one rib or arm when the control part is moved towards the securing device. The securing device comprises a ring-like holding part with inwardly extending holding elements for holding the container inseparably.

The present invention relates to a nebulizer according to the preamble of claim 1, and to a housing part according to the preamble of claim 15.

WO 2006/125577 A2 discloses a nebulizer which comprises, as a reservoir for fluid which is to be atomized or nebulized, an insertable rigid container having an inner bag containing the fluid and a pressure generator with a drive spring for delivering and atomizing the fluid. The container is pre-installed in nebulizer in a delivery state. The pre-installed container is held by a transportation lock unmovable within the housing in the delivery state in order to avoid any undesired opening of the container. Before being used for the first time a lower housing part of the nebulizer is completely closed. Thus, the pre-installed container is opened by a delivery tube piercing a sealing and a septum to fluidically connect to the inner bag of the container. Further, the transportation lock is opened so that the container can move inside the nebulizer back and forth.

By rotating the lower housing part the drive spring can be put under tension and fluid can be sucked into a compression chamber of the pressure generator. Simultaneously, the container is moved into the lower housing part in a stroke movement within the nebulizer and when tensioned for the first time the container may be pierced through its base by a piercing element in the lower housing part to allow venting of the container. After manual operation of a blocking element the drive spring is released and moves the delivery tube into the pressure chamber so that the fluid is put under pressure by the drive spring and is delivered or atomized through a nozzle into a mouthpiece as an aerosol, without the use of propellant gas.

WO 2011/064163 A1 and WO 2012/162305 A1 disclose a similar nebulizer. A container can be inserted into a housing of the nebulizer. The housing is closed by a lower housing part. The container is moving axially forth and back during conveying of the fluid to be nebulized, and during pressure generation and nebulization. The container may be connected inseparably with the housing part by a securing device forming a transportation lock for holding the container unmovable in a delivery state.

Object of the present invention is to provide a nebulizer and a housing part for a nebulizer allowing easy and/or secure operation and/or a compact and/or reliable construction.

The above object is achieved by a nebulizer according to claim 1 or a housing part according to claim 15. Preferred embodiments are subject of the subclaims.

According to one aspect of the present invention, the nebulizer comprises preferably a housing, a container containing the fluid to be nebulized, a securing device forming a transportation lock for holding the container axially unmovable in a non-activated state/delivery state of the nebulizer with closed transportation lock, and a control part for opening the transportation lock, in particular automatically when closing the housing. The control part comprises preferably at least one rib or arm by means of which the transportation lock can be opened when the control part is moved relative to or towards the securing device. This allows a very compact and robust construction wherein the axial dimension can be minimized. The preferred provision of a rib or arm at the control part allows easy and/or secure operation.

Preferably, the rib or arm extends axially. In particular, multiple ribs or arms are provided at the control part or formed by the control part, the ribs or arms extending preferably axially.

Preferably, the rib or arm comprises an actuation or inclined surface for moving or pivoting an associated locking element of the securing device for opening the transportation lock when the control part is moved relative to the securing device, in particular axially towards or into the securing device. This allows reduction of the necessary force and/or use of a longer actuation path for opening the associated locking element and/or the transportation lock.

Preferably, the control part is fixed, in particular by snap-fit, in the position when the transportation lock has been opened or is held open. Preferably, this is realized by a rate catch or snap action between the control part, in particular one or multiple ribs or arms of the control part, on one hand and the securing device, in particular one or more locking elements of the securing device, on the other hand. In particular, the control part or at least one rib or arm thereof comprises a suitable contour, such as a notch, protrusion or shoulder, preferably at the end of an inclined surface, and the securing device or locking element comprises a suitable counter contour, edge or the like for engagement. This allows a very simple realization and/or secure fixing of the control part in the moved position to keep the transportation lock open.

According to another aspect of the present invention, a housing part is provided, in particular for a nebulizer, which comprises preferably a container containing fluid to be nebulized, a securing device forming a transportation lock for holding the container axially unmovable with closed transportation lock, and a control part for opening the transportation lock. Preferably, the control part is constructed as mentioned above, in particular provided with at least one preferably axially extending rib or arm for opening the transportation lock when moving the control part relative to or towards the securing device. Thus, the same advantages result.

Preferably, the securing device holds the container inseparably from the housing part. Thus, the container can be replaced only together with the housing part. This is in particular in advantageous when the housing part comprises a counter device for counting operations or uses of the associated container, in particular the number of doses of fluid dispensed or delivered from the associated container.

According to a further aspect of the present invention, the securing device comprises a preferably ring-like holding part with multiple holding elements for holding the container inseparably from the nebulizer or housing part, and comprises further a base axially spaced from the housing part, wherein the base comprises at least one locking element, preferably multiple locking elements, for holding the container axially unmovable with closed transportation lock. This allows a very reliable and/or robust construction.

Preferably, the holding part and the base are separate parts which are connected, in particular by crimping, snapping or welding. This facilitates or enables production, in particular when the holding part and base are made from steel, most preferably spring steel.

Preferably, the holding part and the base part are inseparably connected with each other.

Preferably, the securing device is snapped into the housing part and/or connected inseparably with the housing part.

In use, the container is preferably moveably in axial direction for dispensing or drawing fluid from the container and/or for pressurization or nebulization of the fluid, in particular in the nebulizer or a pressure generator of the nebulizer.

According to another aspect of the present invention, the nebulizer is preferably provided with such a securing device and/or housing part.

When the housing part of the present invention is connected with or attached to the associated nebulizer and/or the housing of the nebulizer is (completely) closed, the control part is preferably positively or automatically moved relative to or towards or (further) into the securing device (i.e. towards the base part), in particular in axial direction, so that the transportation lock is opened in particular, when or after the container has been fluidically connected and/or opened or vented. Thus, a very easy and/or secure operation can be achieved. In particular, the control part can open the transportation lock in particular the multiple locking elements, by means of one or more ribs or arms with preferably inclined surfaces so that the required force for moving the control part and, in particular, for closing the housing or completely attaching the housing part can be relatively low although the transportation lock or its locking elements can lock the container in the delivery state, i.e. in the non-activated state of the nebulizer and/or with closed transportation lock, with relatively high force and, thus, very securely.

Preferably, the control part is automatically moved or shifted, when the housing part is connected to or with (the rest of) the nebulizer. Then, a contact face, in particular of a housing part or other component, of the rest of the nebulizer abuts at the control part and during the further closing of the nebulizer moves or shifts the control part within or further into the housing part so that the transportation lock is opened.

The above aspects of the present invention and the further aspects described below can be realized independently from each other, and in any combination.

Further advantages, features, characteristics and aspects of the present invention will become apparent from the claims and the following description of a preferred embodiment with reference to the drawings. It shows:

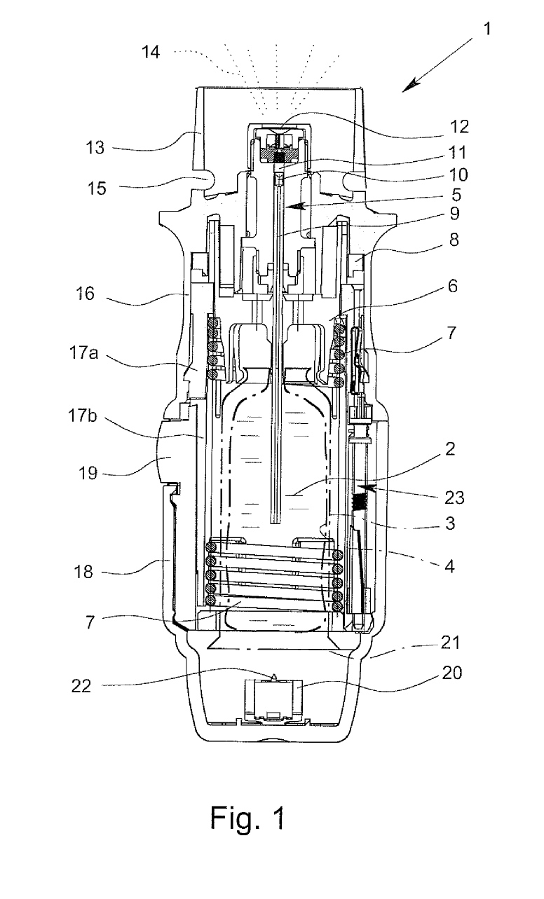

FIG. 1 a schematic section of a known nebulizer in a non-tensioned state;

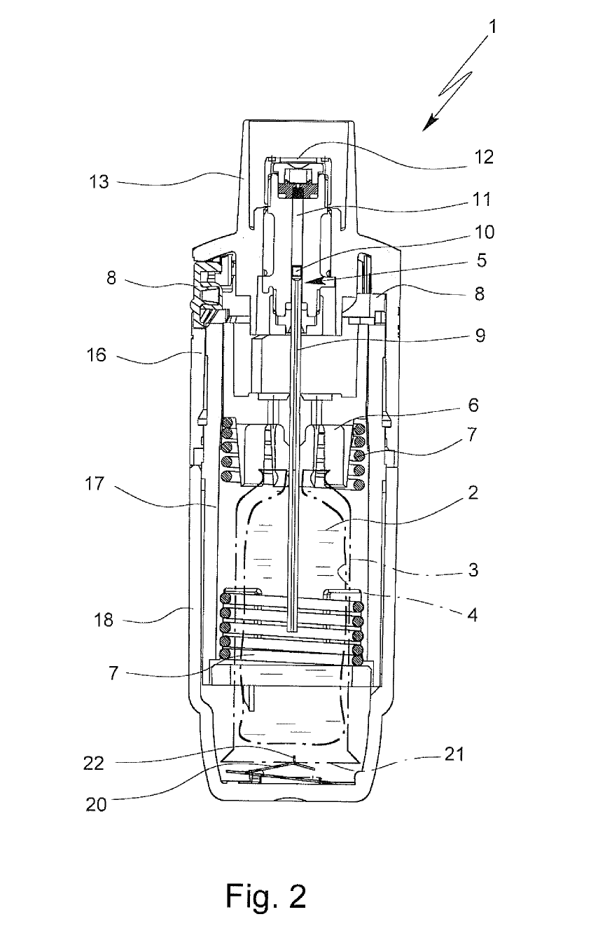

FIG. 2 a schematic section, rotated 90° compared with FIG. 1, of the known nebulizer in a tensioned state;

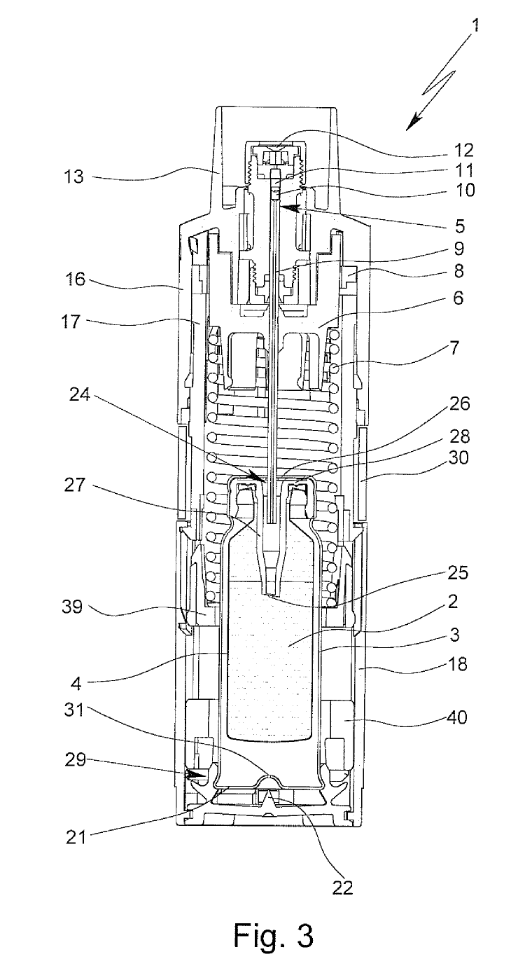

FIG. 3 a schematic section of a nebulizer in a delivery state with a partly closed housing and with a pre-installed, closed container;

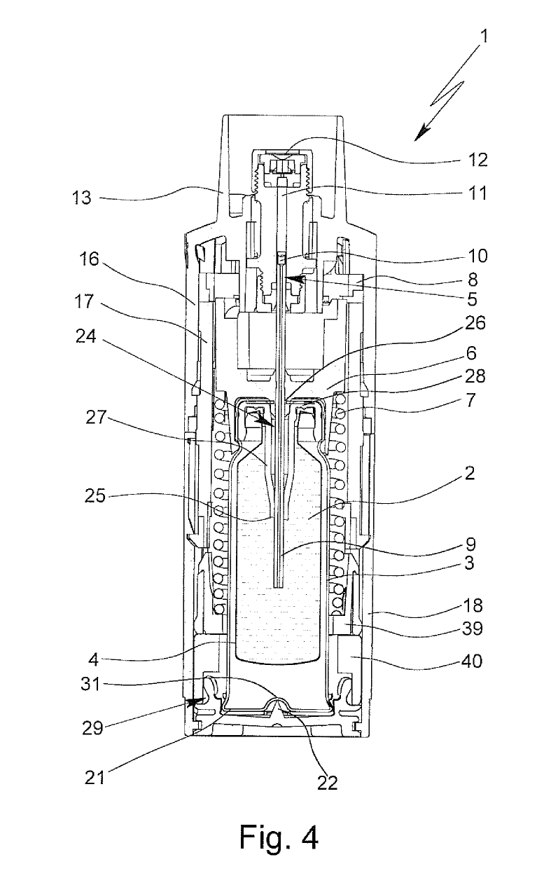

FIG. 4 a schematic section of the nebulizer according to FIG. 3 in an activated, tensioned state with completely closed housing and with opened container;

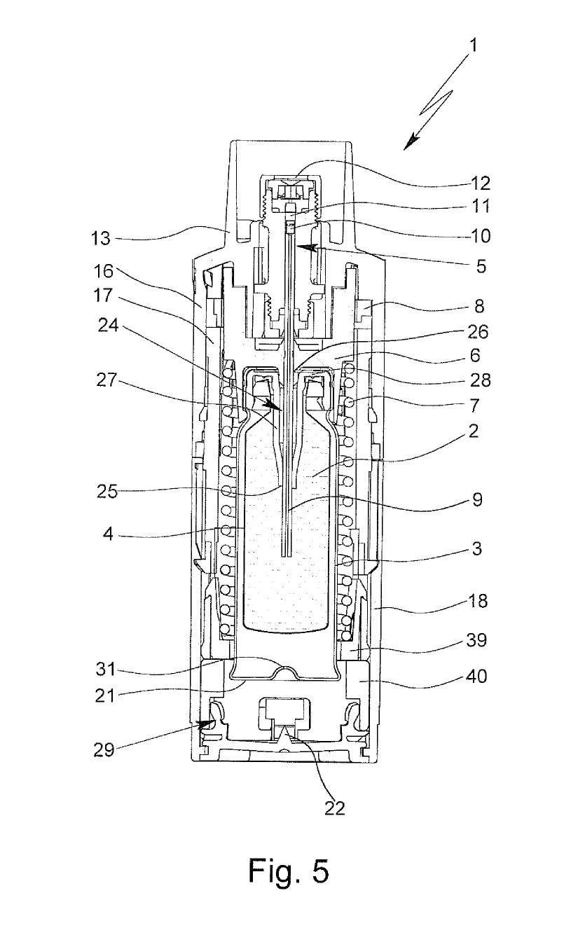

FIG. 5 a schematic section of the nebulizer according to FIG. 4 in a non-tensioned state;

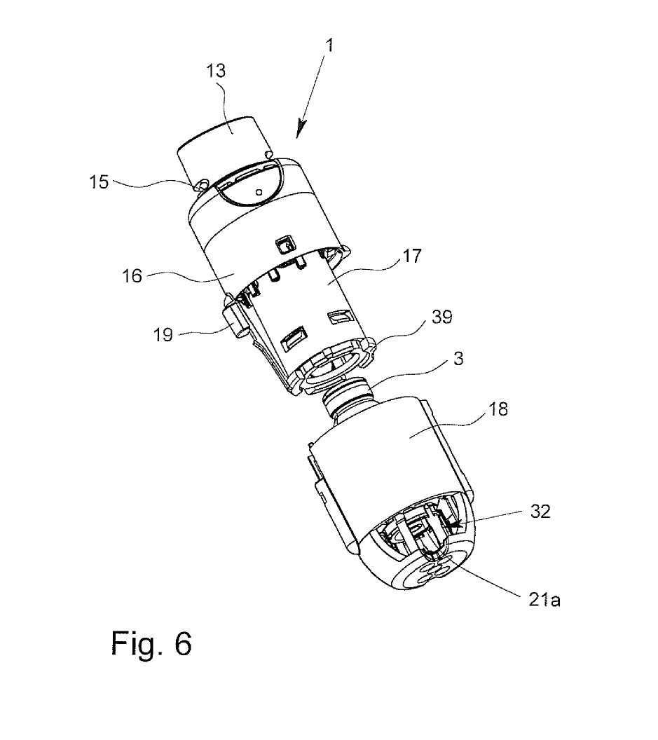

FIG. 6 a schematic perspective view of a nebulizer according to a further embodiment with a separate housing part shown with a partly cut-away portion, the housing part having a securing device holding unmoveably a container of the nebulizer;

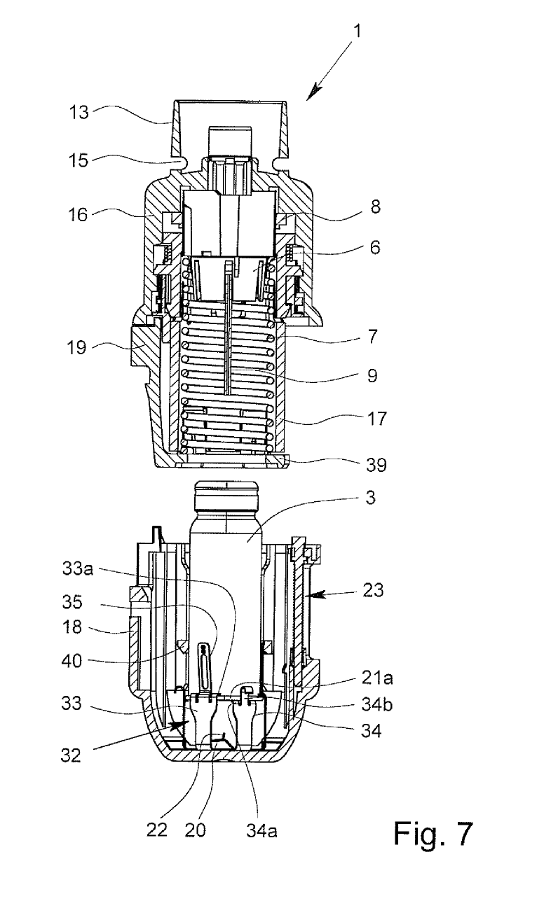

FIG. 7 a schematic section of the nebulizer according to FIG. 6;

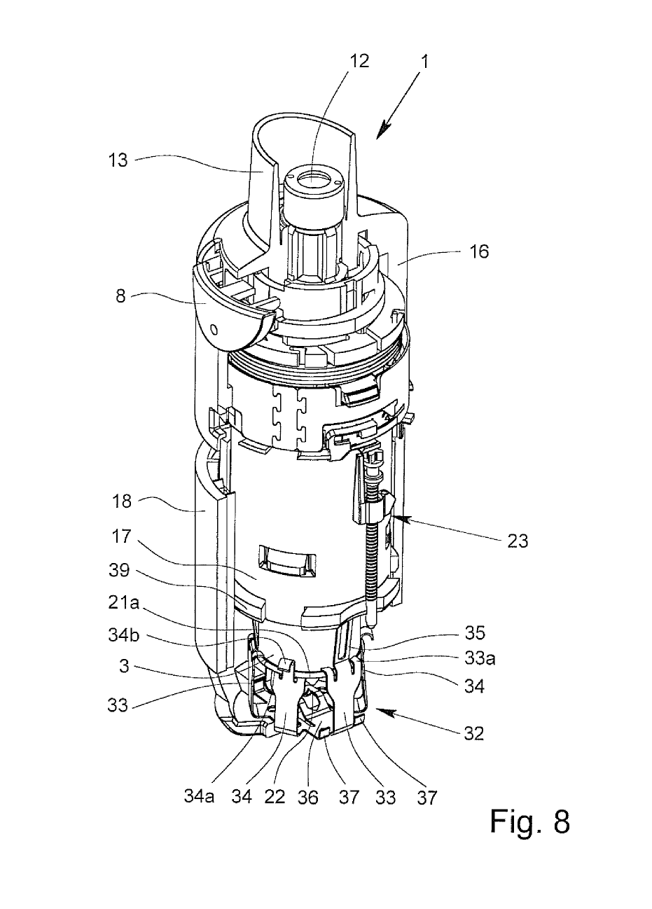

FIG. 8 a schematic side view of the nebulizer according to FIG. 6 with partly mounted housing part and with some cut-away portions, the container being held unmoveably;

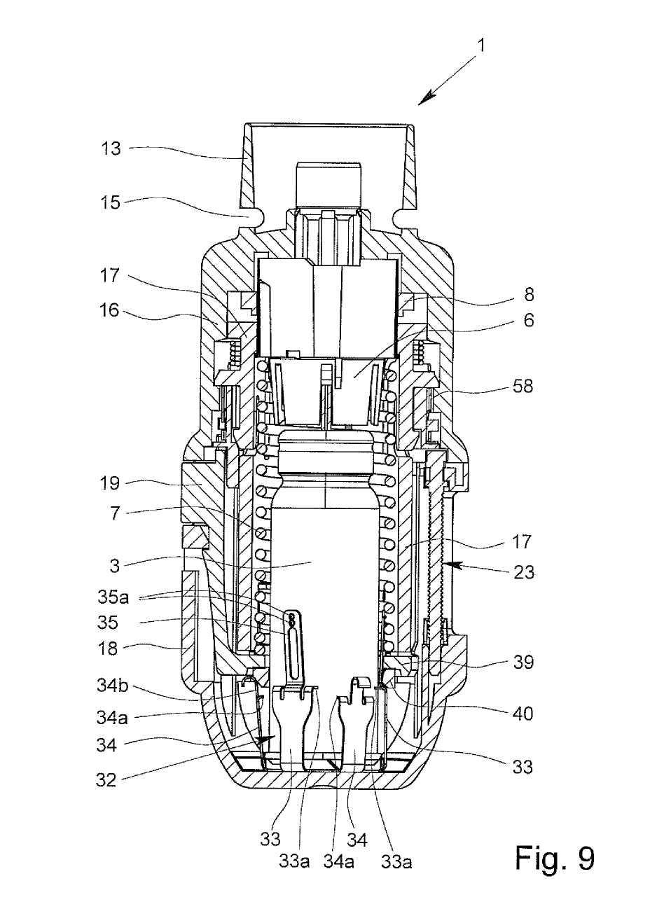

FIG. 9 a schematic section of the nebulizer according to FIG. 6 in the completely closed state with opened securing device so that the container can move axially;

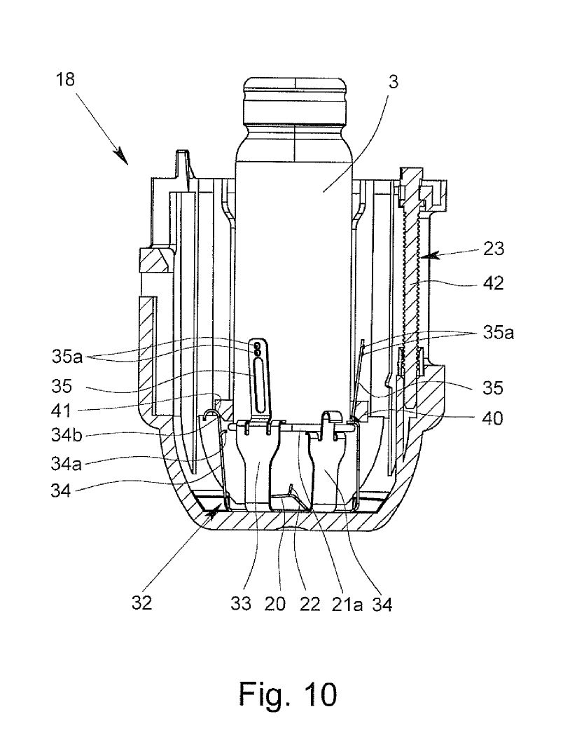

FIG. 10 a schematic section of the housing part with the associated container after use or separation from the nebulizer;

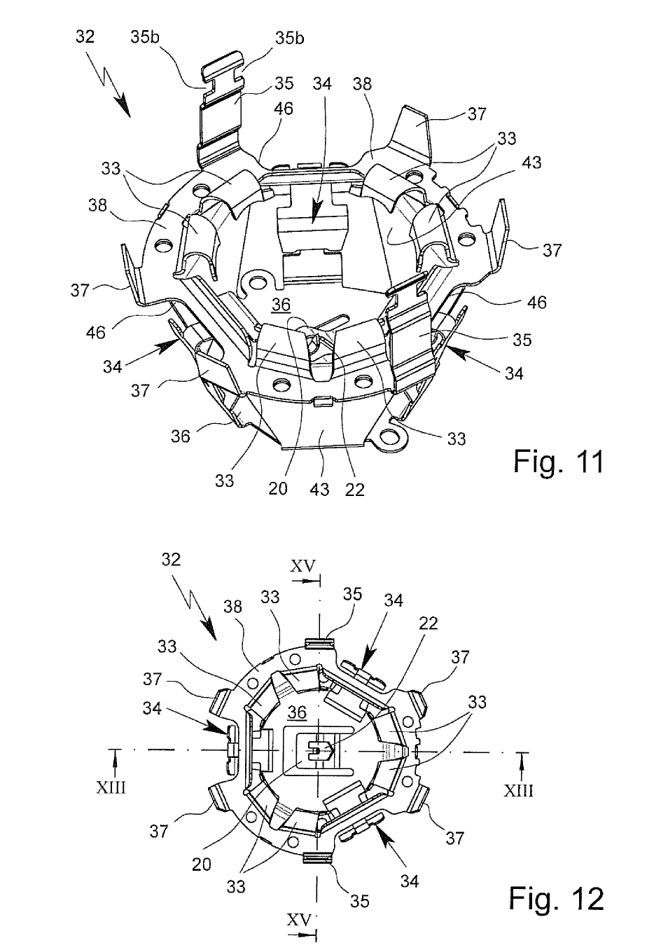

FIG. 11 a perspective view of a securing device of a nebulizer or housing part according to the present invention;

FIG. 12 a top view of the securing device;

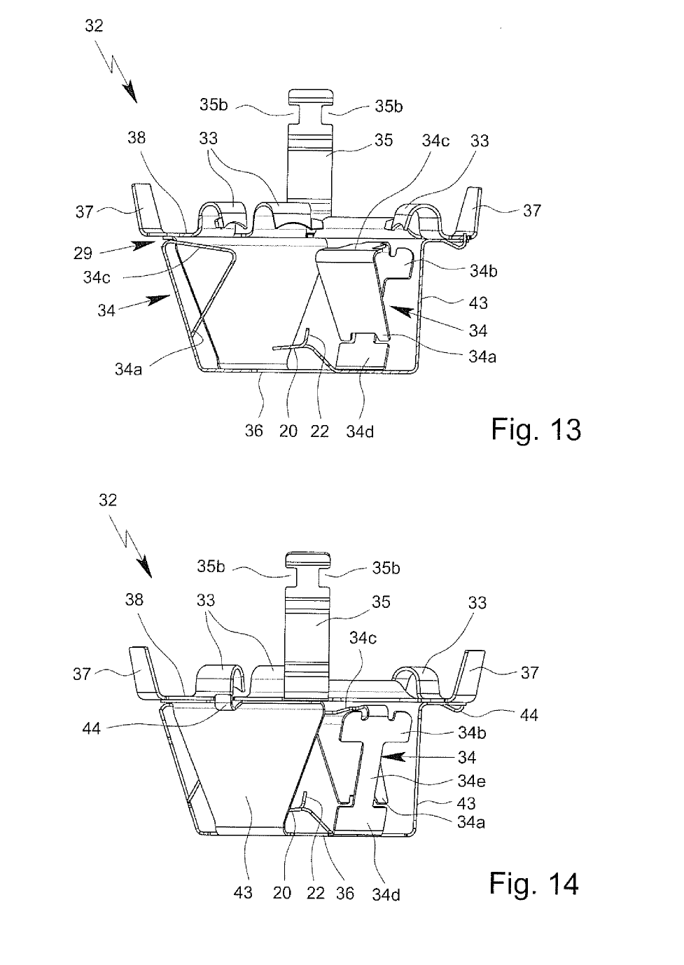

FIG. 13 a schematic section of the securing device along line XIII-XIII of FIG. 12;

FIG. 14 a side view of the securing device in same direction as FIG. 13;

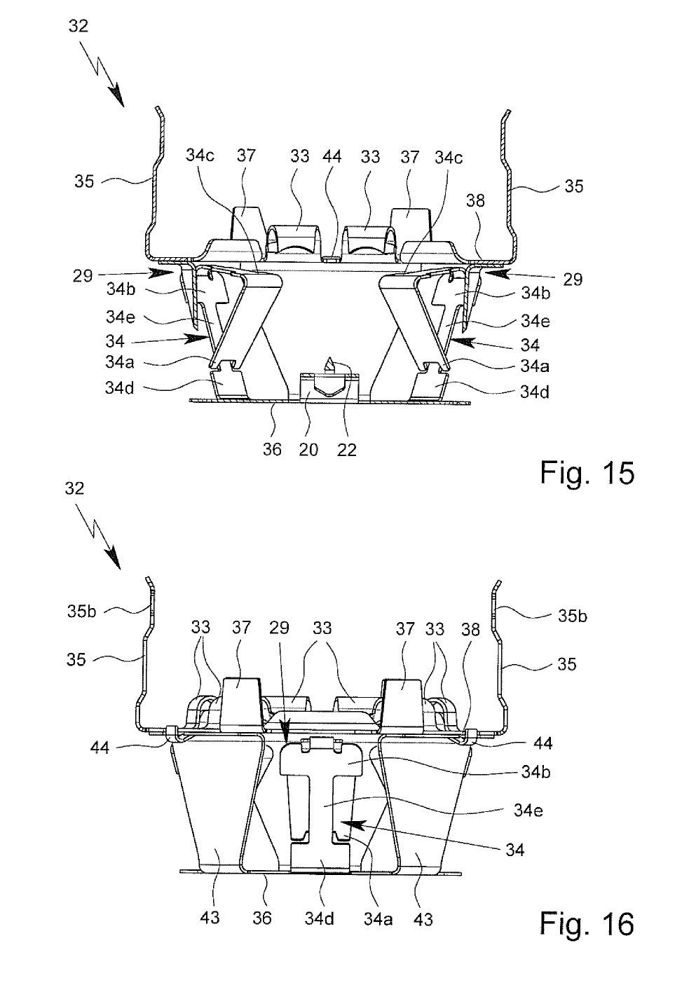

FIG. 15 a schematic section of the securing device along line XV-XV of FIG. 12;

FIG. 16 a side view of the securing device in the same direction as FIG. 15;

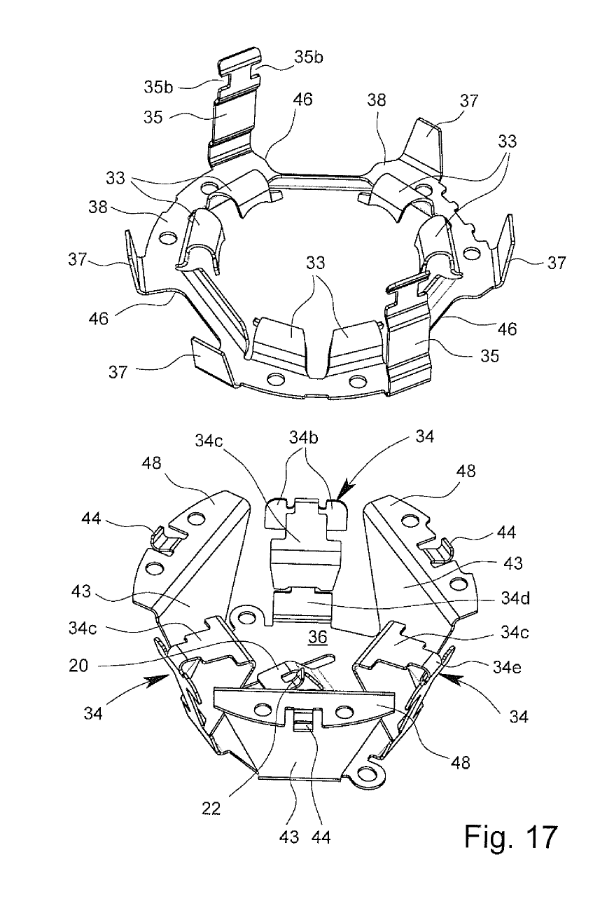

FIG. 17 an exploded perspective view of the securing device;

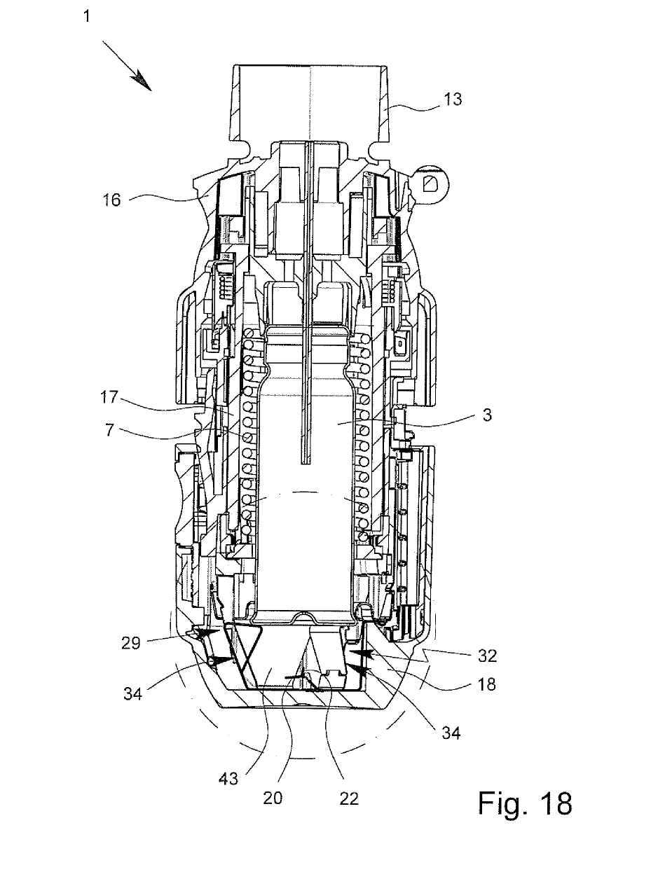

FIG. 18 a schematic section of the nebulizer with partly attached housing part and closed transportation lock;

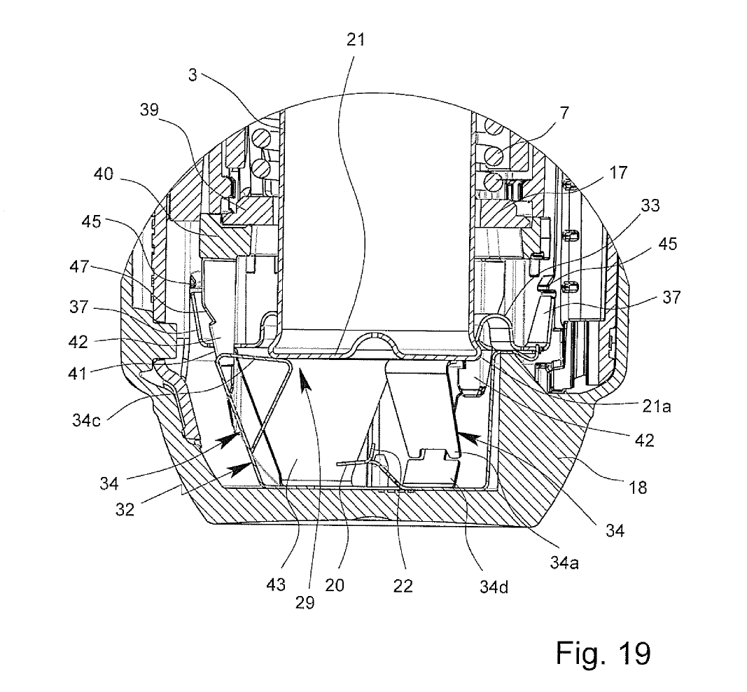

FIG. 19 a partial enlargement of the encircled area of FIG. 18;

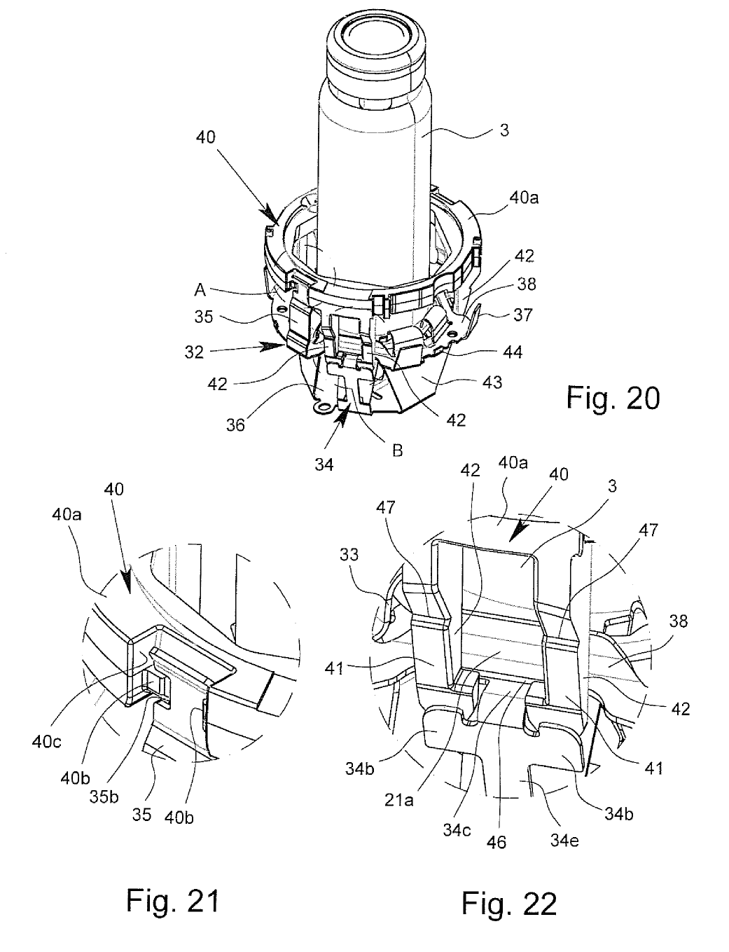

FIG. 20 a schematic perspective view of the container, securing device and control part with closed transportation lock;

FIG. 21 a partial enlargement of the encircled area of FIG. 20 with regard to the cooperation of a locking portion with the control part;

FIG. 22 a partial enlargement of the encircled area of FIG. 20 with regard to the cooperation of arms of the control part with a locking element;

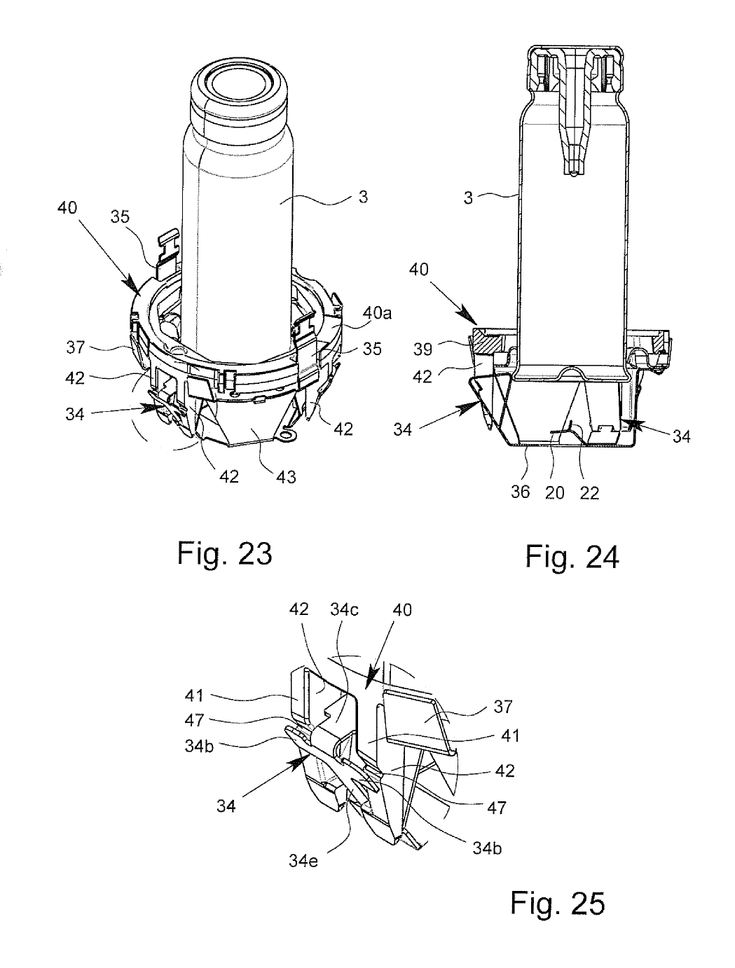

FIG. 23 a schematic perspective view similar to FIG. 20, but with the control part in an intermediate state;

FIG. 24 a schematic section of FIG. 23;

FIG. 25 a partial enlargement of the encircled area of FIG. 23;

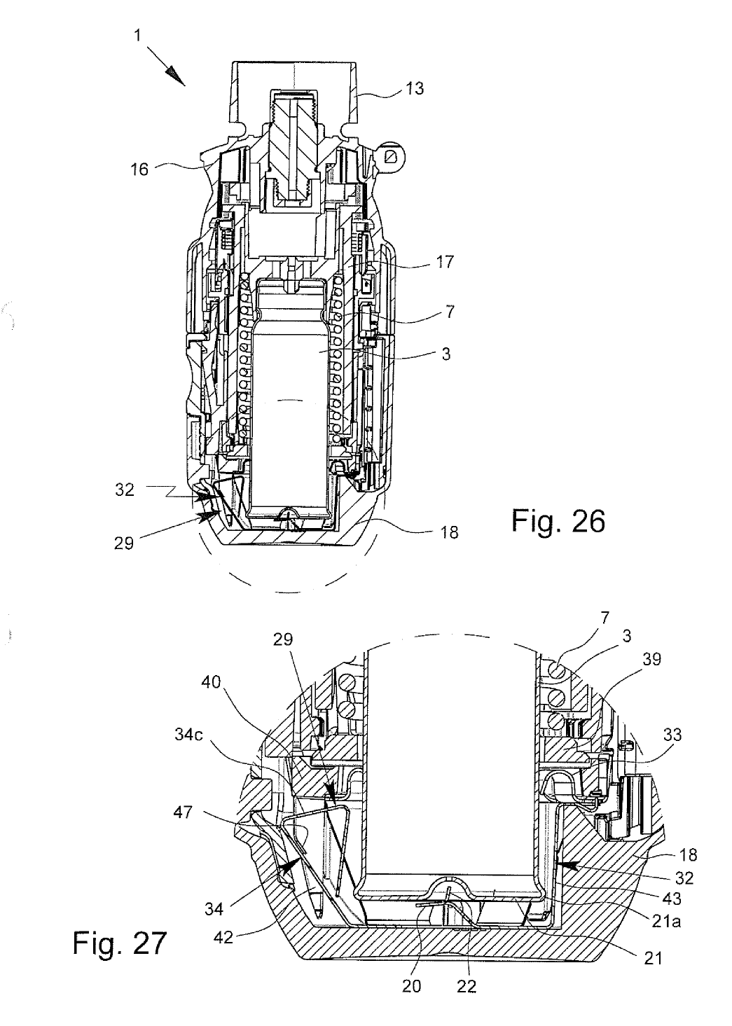

FIG. 26 a schematic section of the nebulizer with partly attached housing part and opened transportation lock;

FIG. 27 a partial enlargement of the encircled area of FIG. 26; and

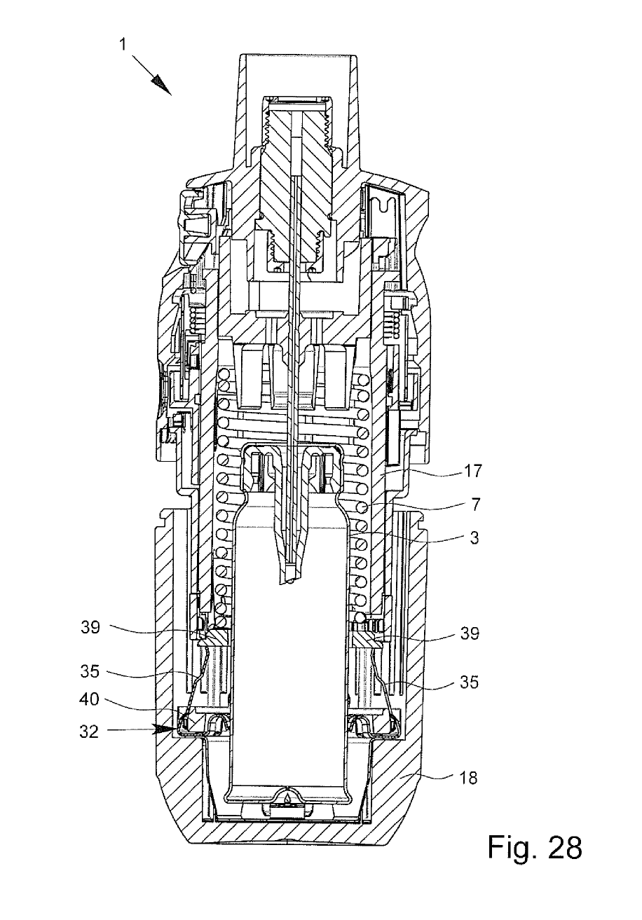

FIG. 28 a schematic section of the re-attachment of the housing part and container to the nebulizer.

In the Figures, the same reference numerals are used for identical or similar parts, resulting preferably in corresponding or comparable properties and advantages, even if the associated description is not repeated.

FIGS. 1 and 2 show a known nebulizer 1 for atomizing a fluid 2, particularly a highly effective pharmaceutical composition, medicament or the like, diagrammatically shown in a non-tensioned state (FIG. 1) and in a tensioned state (FIG. 2). The nebulizer 1 is constructed in particular as a portable inhaler and preferably operates only mechanical and/or without propellant gas.

When the fluid 2, preferably a liquid, more particularly a pharmaceutical composition, is nebulized, an aerosol 14 (FIG. 1) is formed, which can be breathed in or inhaled by a user. Usually the inhaling is done at least once a day, more particularly several times a day, preferably at set intervals, depending on the complain or illness from which a patient is suffering.

The nebulizer 1 is provided with or comprises an insertable or replaceable container 3 containing the fluid 2. The container 3 thus forms a reservoir for the fluid 2, which is to be nebulized. Preferably, the container 3 contains multiple doses of fluid 2 or active substance in particular sufficient to provide up to 200 dosage units or doses, for example, i.e. to allow up to 200 sprays or applications. A typical container 3, as disclosed in WO 96/06011 A1, holds e.g. a volume of about 2 to 20 ml.

It has to be noted that the dose can vary, in particular depending on the fluid 2 or medicament. The nebulizer 1 can be adapted respectively.

Further, the number of doses contained in the container 3 and/or the total volume of the fluid 2 contained in the container 3 can vary depending on the fluid 2 or respective medicament and/or depending on the container 3 and/or depending on the necessary medication or the like.

Preferably, the container 3 can be replaced or exchanged, wherein the number of containers 3, which can be used with the same nebulizer 1, is preferably restricted, e.g. to a total number of four or five containers 3.

The container 3 is preferably substantially cylindrical or cartridge-shaped and once the nebulizer 1 has been opened the container 3 can be inserted therein preferably from below and changed if desired. It is preferably of rigid construction, the fluid 2 in particular being held in a collapsible bag 4 in the container 3. In particular, the container 3 comprises a venting hole 31 which is opened before or during first use.

The nebulizer 1 comprises a delivery mechanism, preferably a pressure generator 5, for conveying and nebulizing the fluid 2, particularly in a preset and optionally in an adjustable dosage amount. The nebulizer or pressure generator 5 comprises preferably a holder 6 for releasably holding the container 3, a drive spring 7 associated to the holder 6, only partly shown, and/or a blocking element 8 preferably in form of or with a button for preferably manual actuation or de-pressing, which blocking element 8 can catch and block the holder 6 and can be manually operated to release the holder 6 allowing drive spring 7 to expand. The nebulizer 1 or pressure generator 5 comprises preferably further a conveying element, such as a conveying tube 9, a non-return valve 10, a pressure chamber 11 and/or an nozzle 12 for nebulizing the fluid 2 into a mouthpiece 13. The completely inserted container 3 is fixed or held in the nebulizer 1 via the holder 6 such that the conveying tube 9 penetrates into the container 3. The holder 6 is preferably constructed so that the container 3 can be exchanged.

When the drive spring 7 is axially tensioned in the tensioning process the holder 6 with the container 3 and the conveying tube 9 are moved downwards in the drawings and fluid 2 is sucked out of the container 3 into the pressure chamber 11 of the pressure generator 5 through the non-return valve 10. In this state, the holder 6 is caught by the blocking element 8 so that the drive spring 7 is kept compressed. Then, the nebulizer 1 is in the so-called activated or tensioned state.

During the subsequent relaxation in the nebulization process after actuation or pressing of the blocking element 8 the fluid 2 in the pressure chamber 11 is put under pressure as the conveying tube 9 with its now closed non-return valve 10 is moved back in the pressure chamber 11, here in the drawings upwards, by the relaxation or force of the drive spring 7 and now acts as a pressing ram or piston. This pressure forces the fluid 2 through the nozzle 12, whereupon it is nebulized into the aerosol 14, as shown in FIG. 1.

Generally, the nebulizer 1 operates with a spring pressure of 5 to 200 MPa, preferably 10 to 100 MPa on the fluid 2, and/or with a volume of fluid 2 delivered per stroke of 10 to 50 μl, preferably 10 to 20 μl, most preferably about 15 μl. The fluid 2 is converted into or nebulized as aerosol 14, the droplets of which have an aerodynamic diameter of up to 20 μm, preferably 3 to 10 μm. Preferably, the generated jet spray has an angle of 20° to 160°, preferably 80° to 100°. These values also apply to the nebulizer 1 according to the teaching of the present invention as particularly preferred values.

A user or patient (not shown) can inhale the aerosol 14, preferably while an air supply can be sucked into the mouthpiece 13 through at least one optional air supply opening 15.

Preferably, the nebulizer 1 or drive spring 7 can be manually activated or tensioned, in particular by actuation of an actuation member.

The nebulizer 1 comprises preferably a housing or (upper) housing part 16 and optionally a biasing or inner part 17 preferably which is rotatable relative thereto (FIG. 2) and/or having an upper part 17a and a lower part 17b (FIG. 1).

The nebulizer 1 comprises preferably an in particular manually operable (lower) housing part or cap 18 releasable fixed, particularly fitted or held onto the inner part 17, preferably by means of a retaining element 19.

Preferably, the housing parts 16 and 18 and/or other parts form a housing of the nebulizer 1. In order to insert and/or replace the container 3, preferably the housing can be opened and/or the housing part 18 can be detached from the nebulizer 1 or its housing. Generally and preferably, the container 3 can be inserted before the housing is closed and/or before the housing part 18 is connected to the housing. Preferably, the container 3 is inserted, opened and/or fluidically connected to the delivery mechanism automatically or simultaneously when (completely) connecting the housing part 18 to the housing/nebulizer 1 and/or when (completely) closing the housing/nebulizer 1.

The actuation member, preferably the housing part 18, can be actuated, here rotated relative to the upper housing part 16, carrying with it or driving the inner part 17. As a result the drive spring 7 is tensioned in the axial direction by means of a gear or transmission (not shown) formed between the inner part 17, in particular its upper part 17a, and the holder 6 and acting on the holder 6. During tensioning the container 3 is moved axially downwards until the container 3 assumes an end position as shown in FIG. 2. In this activated or tensioned state the drive spring 7 is under tension and can be caught or held by the blocking member 8. During the nebulizing process the container 3 is moved back into its original position (non-tensioned position or state shown in FIG. 1) by the drive spring 7. Thus the container 3 executes a lifting or stroke movement during the tensioning process and during the nebulizing process.

The housing part 18 preferably forms a cap-like lower housing part and fits around or over a lower free end portion of the container 3. As the drive spring 7 is tensioned the container 3 moves with its end portion (further) into the housing part 18 or towards the end face thereof, while an aeration means, such as an axially acting spring 20 arranged in the housing part 18, comes in contact with base 21 of the container 3 and pierces the container 3 or a base seal thereon with a piercing element 22 when the container 3 makes contact with it for the first time, to allow air in or aeration.

The nebulizer 1 comprises preferably a counter device 23, which counts in particular actuations of the nebulizer 1, preferably by detecting its tensioning or the rotation of the inner part 17 relative to the upper part 16 or housing. Preferably, the counter device 23 or an associated lock locks the (further) actuation or use of the nebulizer 1, e.g. blocks further rotation of the housing part 18/inner part 17 and, thus, tensioning of the nebulizer 1 or its drive spring 7 and/or blocks actuation of the blocking element 8, when a certain number of actuations or operations or discharged doses has been reached or exceeded.

A preferred construction and mode of operation of the inhaler or nebulizer 1 will now be described in more detail with reference to FIGS. 3 to 5, but emphasizing only essential differences from the nebulizer 1 according to FIGS. 1 and 2. The remarks relating to FIGS. 1 and 2 thus apply preferably accordingly or in a similar manner, while any desired combinations of features of the nebulizer 1 according to FIGS. 1 and 2 and the nebulizer 1 described below are possible.

Preferably, the container 3 is pre-installed. This can be realized in particular as shown in WO 2006/125577 A2 or as described in the following.

FIG. 3 shows the nebulizer 1 in a delivery state with preferably pre-installed container 3, which is still closed. In this state, the housing of the nebulizer 1 is not completely closed, in particular the housing part 18 is not completely pushed on the inner part 17. FIGS. 4 and 5 show the nebulizer 1 in an activated state with the housing completely closed and with the container 3 opened. In FIG. 4, the nebulizer 1 or drive spring 7 is tensioned, i.e. the container 3 is in its lower position. FIG. 5 shows the nebulizer 1 in a non-tensioned state, e.g. after dispensing or nebulizing of one dose of the fluid 2; the container 3 is in its upper position.

The container 3 comprises a fluid outlet 24 for outputting the fluid 2 to be dispensed. In particular, the fluid outlet 24 allows a fluidic connection between the container 3 or its bag 4 on one hand and the nebulizer 1, its pressure generator 5 or the conveying element on the other hand.

The fluid outlet 24 has an inner closure 25 that is preferably formed by a septum, a membrane, a plastic seal or the like and/or is provided inside the container 3. Optionally, a second or outer closure 26 can be provided such that successive opening is possible by means of one common element, in particular the conveying element or conveying tube 9 or the like, and/or by piercing.

Preferably, the first or inner closure 25 is formed or supported by a closure part 27 extending from the outlet or head end of the container 3 into the container 3 or bag 4. The second or outer closure 26 is preferably located adjacent to the head or axial end of the container 3 and/or held or connected to a flange 28, which can be formed by the closure part 27 or any other suitable part. However, other constructional solutions are possible.

In the delivery state according to FIG. 3, the container 3 has been pre-installed, i.e. inserted into the nebulizer 1. However, the container 3 or its fluid outlet 24 is not yet opened. In particular, the second closure 26 is already opened, but not the first closure 25. This is achieved in particular in that the housing of the nebulizer 1 is closed only partly, i.e. not completely, in the delivery state.

In particular, the container 3 is attached to or held by or secured in the housing part 18, in particular by a transportation lock 29, which is preferably arranged within or at the housing part 18. The transportation lock 29 holds the container 3 preferably temporarily, in particular before attaching the housing part 18 to the nebulizer 1 and/or in the delivery state. In particular, the transportation lock 29 holds the container 3 fixed during the fluidic connection of container 3 and/or during the mechanic connection of container 3, here with holder 6. Preferably, the transportation lock 29 holds the container 3 fixed during opening, in particular piercing, the container 3.

In the delivery state, in which the nebulizer 1 can be shipped or delivered to the user or is still packed, the nebulizer 1 or the housing part 18 is preferably secured, in particular by means of a securing member 30, e.g. a banderole, such that the container 3 and/or housing part 18 are held sufficiently spaced from the nebulizer 1 or upper housing part 16 and/or prevented from being completely closed or completely inserted or pushed on the conveying element or tube 9, the housing or inner housing part 17 or the like and/or such that (complete) opening of the container 3, namely of the first closure 25, is prevented.

Once the security member 30 has been removed a user (not shown) can push the housing part 18 fully on in the axial direction and thereby open the container 3, i.e. first closure 25, by inserting the conveying element or conveying tube 9. FIGS. 4 and 5 show this activated state with the housing part 18 pushed fully on and/or the container 3 open (fluidically connected to the nebulizer 1 or its pressure generator 5 or the conveying element or tube 9).

FIG. 4 shows the nebulizer 1 or container 3 in the activated state, the container 3, i.e. first closure 25, is open, i.e. the container 3 or its fluid 2 is fluidically connected to the nebulizer 1 or its pressure generator 5, and the housing part 18 has been pushed fully on in the axial direction. In order to bring the holder 6 into (complete) engagement with the container 3 at the head end and then be able to move the container 3 back and/or forth for the suction/tensioning and pressing strokes, it may be necessary to tension the nebulizer 1 or it drive spring 7 for the first time. During this tensioning process the holder 6 is moved together with the conveying tube 9 axially towards or into the housing part 18, thus bringing the holder 6 into (complete) engagement with the container 3 and preferably also moving or pressing the container 3 against the piercing element 22 in the region of the base of the housing part 18 and thereby piercing or opening a venting hole 31 in the container base 21. FIG. 4 shows the nebulizer 1 in this tensioned and activated state. The holder 6 is engaged with the container 3 and the conveying tube 9 has been fully inserted into the container 3.

FIG. 5 shows the nebulizer 1 in the relaxed, non-tensioned state, i.e. after atomization or discharge of a dose of the fluid 2. The holder 6 and the container 3 are in the upper position. The holder 6 is still engaged with the container 3 and remains engaged during the further uses of the nebulizer 1. Further, the container 3 is still open and fluidically connected, i.e. the nebulizer 1 remains activated.

To prevent unwanted opening of the container 3, particularly of the first closure 25, in the delivery state of the nebulizer 1, and/or to prevent (axial) movement of the container 3 relative to the associated housing part 18 before complete closing of the nebulizer 1, preferably the transportation lock 29 is provided. By frictional, forcible or interlocking engagement, for example, the transportation lock 29 prevents the container 3 from undesirably moving axially.

Preferably, the opening of the transportation lock 29 occurs automatically when closing the nebulizer 1 or its housing completely, i.e. when snapping or pushing on the housing part 18 completely towards the upper housing part 16. During this (preferably linear, axial or telescopic) closing movement, the transportation lock 29 is opened and the container 3 released in axial direction preferably during or after piercing or opening the container 3 and/or preferably during only a last part of the movement and/or just little before the final completely closed position is reached or just when the final completely closed position is reached.

During the closing movement in which preferably parts 17 and 18 are joined, the transportation lock 29 is preferably opened by the direct or indirect interaction with or actuation by the housing of the nebulizer 1, the inner part 17 or its lower part 17b or the like. Preferably, the container 3 and/or first closure 25 are opened as well as the transportation lock 29 by means of a common actuation and/or component, here the closing movement of the nebulizer 1 or its housing or bottom part 18.

FIGS. 4 and 5 show the transportation lock 29 in the open position, i.e. wherein the container 3 is free to move axially.

In the following, a further embodiment of the nebulizer 1 will be described in more detail with reference to FIGS. 6 to 10, wherein only essential differences from the nebulizer 1 described above or shown in FIGS. 1 to 5 will be emphasized or described. Thus, the remarks relating to FIGS. 1 to 5 apply preferably accordingly or in a similar manner, while any desired combinations of features are possible.

FIG. 6 shows the nebulizer 1 in a perspective side view with not yet mounted, i.e. separated (lower) housing part 18 (partly cut open for illustration purposes) with associated container 3. The container 3 has not been inserted or pre-installed in the nebulizer 1 yet. With other words, the nebulizer 1 has not been assembled yet or is not in the preferred delivery state yet.

FIG. 7 shows the nebulizer 1 in a schematic section as well as the container 3 and housing part 18 which are still separated from the (upper part of the) nebulizer 1.

The nebulizer 1 or its housing or housing part 18 comprises preferably a securing device 32 which may have different functions. The securing device 32 may hold the container 3 such that the container 3 is moveable back and forth within the completely closed housing for conveying the fluid 2, pressure generation and/or nebulization, wherein the securing device 32 may ensure that the container 3 is inseparable from the housing or housing part 18. Thus, only complete replacement of the housing part 18 together with the respective container 3 is possible. Alternatively or additionally, the securing device 32 may form the transportation lock 29. Alternatively or additionally, the securing device 32 may prevent that the used container 3 and/or used housing part 18 can be (re)connected to or used with the nebulizer 1 once more. Especially the securing device 32 may prevent that any used container 3 and/or used housing part 18 can be connected to a nebulizer once after it has already been detached from the nebulizer and/or the inner part 17 of the nebulizer.

When the securing device 32 or transportation lock 29 is closed, the container 3 is held or counter-beared for opening by inserting the conveying element or tube 9, preferably wherein a press-fit is formed between the conveying element or tube 9 and the container 3 or closure part 27, and/or for (completely) connecting the container (head) to the holder 6. With other words, the transportation lock 29 or securing device 32 form preferably a counter-bearing for the container 3 during closing of the nebulizer 1.

When the securing device 32 or transportation lock 29 is closed, the container 3 is held spaced from the piercing element 22.

The securing device 32 is preferably located or arranged or fixed at or in the housing part 18 as shown in FIGS. 6 and 7.

Preferably, the securing device 32 comprises or consists of a metal and/or stamped part and/or consists of a single, unitary part. The securing device 32 may consist of two or more parts, preferably connected by means of gluing, crimping, riveting, welding, or the like.

Preferably, the securing device 32 is made of steel, in particular spring steel. Preferably, the securing device 32 is produced from sheet material by cutting, stamping or the like and/or by bending. Preferably, the securing device 32 or a part thereof forms a cage, in particular encompassing the container 3 or an end portion thereof, in particular the container base 21.

Preferably, the securing device 32 comprises holding elements 33 and/or locking elements 34. The elements 33 and/or 34 are preferably designed like arms, fingers, leaves or the like. In particular, the elements 33 and 34 are alternately distributed over the circumference of the container 3. Preferably, the securing device 32 comprises multiple holding elements 33 and multiple locking elements 34, in particular three or more holding elements 33 and three or more locking elements 34. Preferably, the elements 33 and 34 extend at least essentially axially and/or in the direction of the back and forth movement of the container 3 and/or in the direction of the longitudinal or main extension of the nebulizer 1 or main dispensing direction of the aerosol 14.

Preferably, the elements 33 and 34 are held by or connected with a base 36 of the securing device 32, as shown in FIG. 8. FIG. 8 shows the nebulizer 1 in a schematic side view with already partly mounted housing part 18 and with some cut-away portions. The transportation lock 29 or securing device 32 is still closed or locked, i.e. the container 3 is still securely held so that it cannot axially move (axially means in the direction of the back and forth or stroke movements).

Preferably, the securing device 32 or base 36 comprises or holds the piercing element 22 for piercing the container 3, i.e. opening the container base 21 or its venting hole 31 or a respective sealing of the container 3 or the like in the activated and tensioned state, i.e. when the container 3 reaches its lower end position. In the shown and preferred embodiment, the piercing element 22 is formed by a respective bending of a spring 20 of the securing device 32 or its base 36. The spring 20 can support or facilitate the (complete or final) connection of the container 3 to the holder 6.

The securing device 32 or base 36 comprises preferably at least one or multiple fixing portions 37 for fixing the securing device 32 at or in the nebulizer 1 or housing or housing part 18. In particular, the fixing portions 37 may fix the securing device 32 when it is pressed into the housing part 18 by cooperating with the sidewall of the housing part 18. However, it is also possible to overmold the securing device 32, its base 36, the fixing portions 37 or the like. Moreover, the securing device 32 could be connected with the housing part 18 or the like in any other suitable manner, in particular, by a separate fixing member, by gluing or the like.

As already mentioned, the securing device 32 preferably forms the transportation lock 29 for holding the container 3 unmovable in the housing or housing part 18 in the delivery state of the nebulizer and/or before attaching the housing part 18 to the nebulizer 1. In this situation (in particular in the delivery state), the container 3 or a preferably radially protruding and/or circumferentially extending part or container edge 21 a thereof, preferably formed at the container base 21, is held preferably in a form-fit manner and/or between the holding elements 33 and locking elements 34, in particular between respectively formed or bent end portions 33a and 34a of the elements 33 and 34, respectively, as shown in FIGS. 6 to 8.

In the shown embodiment, the container 3 and/or container edge 21a is caught between the end portions 33a and 34a, preferably alternatively. The holding elements 33 and/or end portions 33a grip or extend over the container edge 21 a, and the locking elements 34 or its end portions 34a grip or extend under the container edge 21a, so that the container edge 21a and container 3 are securely held in between, in particular by form-fit, preventing any axial movement of the container 3 relative to the securing device 32 and relative to the associated housing part 18 in this state or delivery state, i.e. with locked transportation lock 29/securing device 32.

Preferably, the end portions 33a and/or 34a are formed like claws or the like and/or extend preferably radially inwardly.

Preferably, the elements 33 and/or 34 can flex with its free ends radially outwardly.

Preferably, the securing device 32 is designed such that the associated container 3 can be connected with the securing device 32 by a respective axial force or movement, wherein the elements 33 and/or 34 flex preferably automatically outwardly as required to receive the container 3 in the locked position as shown in FIGS. 6 to 8. However, if necessary, a suitable tool (not shown) or the like could be used alternatively or additionally for assembly if necessary.

For example, the ends of the end portions 33a could be inclined such that the container 3 may be inserted into or connected with the securing device 32 by a respective axial movement so that the holding elements 33 flex outwardly to allow passing of container edge 21a.

Preferably, the holding elements 33 or its end portions 33a prevent separation of the container 3 from the securing device 32 and, thus, from the associated housing part 18 or the like.

In the present embodiment, the holding elements 33 extend preferably above the end portions 33a and/or form or comprise preferably arm-like guiding and/or locking portions 35. These axial extensions and/or these portions 35 extend axially beyond the end portions 33a and/or may cooperate with the container 3 or its container edge 21 a during axial assembly of the container 3 with the securing device 32 such that the holding elements 33 are flexed sufficiently outwardly so that the container edge 21 a can pass the end portions 33a and the container base 21 can be seated on the end portions 34a of the locking elements 34.

The locking elements 34 preferably comprise actuation portions 34b at its free ends extending axially beyond the end portions 34a. The actuation portions 34b may radially guide the container 3 and/or facilitate insertion of the container 3 or its container edge 21a between the free ends of the locking elements 34 although the locking elements 34 are preferably radially inwardly biased as well as the holding elements 33.

When, the container 3 is held with its container edge 21a between the end portions 33a and 34a, the transportation lock 29/securing device 32 is closed, i.e. the container 3 cannot move axially within the housing part 18 or nebulizer 1.

For opening the transportation lock 29 or securing device 32, the locking elements 34 and/or its end portions 34a are flexed preferably radially outwardly so that the container 3 can freely move axially, in particular restricted such that the container edge 21 a can only move axially within the securing device 32 and/or that the axial movement is restricted (in the drawings upwardly) by the holding elements 33 or its end portions 35 and/or such that the container 3 cannot be separated from the securing device 32. This opening of the transportation lock 29 or securing device 32 will take place when activating the nebulizer 1, when using the nebulizer 1 for the first time and/or when completely closing the nebulizer 1. Then, the container 3 can axially move, in particular back and forth and/or with its container edge 21 a between the end portions 33a and the piercing element 22 in the present embodiment. This situation is schematically shown in the schematic section according to FIG. 9 which shows the nebulization with closed housing or housing part 18 and with opened transportation lock 29/securing device 32.

In FIG. 9, the container 3 is shown in its lower position similar to FIG. 4, in particular, wherein the container base 21 is in contact with the piercing element 32.

However, the nebulizer 1 is not shown in the tensioned state, i.e. the holder 6 is not in its lower position, i.e. the holder 6 is not yet connected with the upper end or head of the container 3. Normally, the holder 6 would be connected to the container 3 in this situation with the container 3 in the lower position. With other words, usually the nebulizer 1 or drive spring 7 would be tensioned in the situation with the container 3 being in the lower position.

Preferably, the container is finally or completely or correctly connected to holder 6 when tensioning the nebulizer 1 or its drive spring 7 for the first time after completely closing the nebulizer 1. However, it is generally also possible that the nebulizer 1 is in the tensioned state, i.e. the drive spring 7 is already tensioned and the holder 6 is in the lower position, before or during (first) assembly with lower housing part 18. Consequently, the holder 6 should directly connect with the container 3 when completely closing the housing part 18, and the situation shown in FIG. 9 should normally not occur.

In FIG. 9, the end portions 34a are moved radially outwardly in order to open the transportation lock 29 or securing device 32. This is achieved preferably by flexing the locking elements 34 radially outwardly. This can be achieved in particular by acting on the actuation portions 34b.

Preferably, the transportation lock 29 is opened or the locking elements 34 are flexed outwardly when completely closing the nebulizer 1 or its housing part 18, in particular by cooperation with or abutment of the inner part 17, its lower part 17b, a retaining part 39 and/or a control part 40. The retaining part 39 is preferably arranged at the upper housing part 16 or inner part 17. The control part 40 is preferably arranged in the lower housing part 18.

The retaining part 39 is connected to the lower or free end of the inner part 17 or its lower part 17b in order to hold, bear or support an end (the lower end) of the drive spring 7. FIGS. 3 to 5 show a preferred construction of the retaining part 39. It is preferably formed as a ring and/or provided with hooks or the like for inter-connection with the inner part 17. In the preferred embodiment, the retaining element 19 forms a unitary component or portion of the retaining part 39. However, other constructional solutions are possible.

In the embodiment shown in FIGS. 3 to 5, a preferably ring-like control part 40 opens the transportation lock 29, in particular flexible arms of the transportation lock 29, to allow axial movement of the container 3. This control part 40 keeps the transportation lock 29 or its flexible arms open when the nebulizer 1 or its housing part 18 has been completely closed. The control part 40 is pushed axially downwards by abutment of the inner part 17 or retaining part 39 within the housing part 18 when completely closing the nebulizer 1.

In the embodiment shown in FIGS. 6 to 10 and particular in FIG. 9, the nebulizer 1, housing part 18 or securing device 32 comprises the preferably ring-like control part 40 for opening the transportation lock 29/securing device 32 or its locking elements 34. In particular, the control part 40 is pushed axially downwards when closing the nebulizer 1 so that it is moved between the locking elements 34 or its actuation portions 34b and exerts an axial force radially outwards. Preferably, an inclined plane converts the axial or closing movement into an opening or radial movement which forces the actuation portions 34b radially outwardly when the control part 40 is forced axially downwardly, here by abutment of the retaining part 39, in particular in the axial end position shown in FIG. 9.

Preferably, the control part 40 and/or locking elements 34 or actuation portions 34b comprise a respectively inclined actuation surface 41 or the like to convert the axial closing movement or movement of the control part 40 into the desired radial opening movement of the locking elements 34 or actuation portions 34b and, thus, of the end portions 34a to open the transportation lock 29, in particular when the housing has been completely closed or when the housing part 18 has been pushed completely on the nebulizer 1.

However, other constructional solutions are possible to realize opening of the transportation lock 29 or securing device 32 or its locking elements 34 or end portions 34a when closing the nebulizer 1 or its housing parts 18.

In the preferred embodiment the control part 40 serves alternatively or additionally another purpose. Namely, the control part 40 prevents the locking portions 35 from moving radially apart or flexing radially outwards before the nebulizer 1 is assembled with its housing part 18 for the first time.

As already mentioned, the securing device 32 prevents preferably that a container 3 can be connected to or used with the nebulizer 1 once more. In particular, the securing device 32 can prevent that a used housing part 18 or used container 3 can be reconnected to the nebulizer 1 once it has been detached from the nebulizer 1. Thus, the securing device 32 prevents any undesired reuse of the container 3 and/or housing part 18 with its preferably inseparable container 3.

In the present embodiment, the undesired reuse is prevented in that the locking portions 35 force apart or move apart or radially and/or outwards at least after the used container 3 and/or housing part 18 has been detached from the nebulizer 1 such that the used container 3 and/or housing part 18 cannot be connected to or used with the nebulizer 1 once more. Preferably, the locking portions 35 are biased such that the locking portions 35 force apart or move radially and/or outwards after release.

In the preferred embodiment, the locking portions 35 are held together or held against moving apart, radially and/or outwards by the control part 40 (schematically indicated in FIG. 7) before the container 3 and the associated housing part 18 have been connected to the nebulizer 1 for the first time. In this pre-assembly state, the control part 40 is located preferably near the free ends of the locking portions 35 and/or it encompasses that locking portions 35 such that locking portions 35 are held sufficiently close together to be inserted with its free ends within the retaining part 39 and/or drive spring 7 when pushing the housing part 18 axially onto the nebulizer 1 or its inner part 17, in particular lower part 17b.

The control part 40 may cooperate with the locking portions 35 or protrusions 35a thereof (shown in FIG. 9) preferably such that the control part 40 is held by a preferably radial engagement and/or frictional force in its (upper) position holding the locking portions 35 or holding elements 33 together in the pre-assembly state. Later during assembly, in particular during complete closing of the housing or pushing on the housing part 18, the locking portions 35 are moved into the retaining part 39 and drive spring 7, while the control part 40 is moved axially downwards or towards the securing device 32, the container base 21 and/or bottom part of the end of the housing part 18. Then, the end position or completely assembled position is reached as shown in FIG. 9. In this state, the radially biased locking portions 35 are held together by the drive spring 7 as the control part 40 does not hold the locking portions 35 together any more.

Preferably, the control part 40 has opened the transportation lock 29 or locking elements 34 in the last part of the closing movement or just when completely closing the nebulizer 1 as already mentioned.

The schematic section of FIG. 10 shows the housing part 18 together with its associated container 3 after it has been used and separated from the nebulizer 1. The control part 40 remains preferably in its lower position. The transportation lock 29 is (still) open. The container 3 is shown in its upper position where it is held by the end portions 33a of the holding elements 33 when detaching the container 3 from the nebulizer 1, in particular from the holder 6 and the conveying element or tube 9.

FIG. 10 shows that the locking portions 35 have been forced apart, in particular due to its biasing or elastic force, here moved radially outwardly with its free ends in particular due to its preferably radial biasing or elastic force. This forced apart position of the locking portions 35 blocks reconnection of the container 3 and/or housing part 18 and/or securing device 32 with the nebulizer 1. Thus, the already used container 3 cannot be reused. Thus, misuse of the container 3 or nebulizer 1 can be prevented.

The control part 40 may additionally secure the holding elements 33 or its end portions 33a against radial opening when the control part 40 is in its lower position as shown in FIGS. 9 and 10. In this case, the control part 40 contacts the holding elements 33 preferably on the outer side to prevent or restrict any outward flexing. Thus, the securing device 32 or its holding elements 33 or end portions 33a are secured against opening so that the container 3 or its container edge 21a is securely held within the securing device 32 or the cage formed by the securing device 32 or holding elements 33.

In the preferred embodiment, the counter device 23 is arranged preferably at or in the housing part 18 as schematically shown in FIGS. 7 to 10.

The counter device 23 counts the actuations or operations of the nebulizer 1 or the discharged doses, preferably for the respective container 3.

Preferably, the counter device 23 counts actuations or operations by detecting the rotation of the inner part 17 relative to the upper part 16 of the housing. With other words, the counter device 23 may count the tensioning the nebulizer 1 or its drive spring 7. However, other constructional solutions are possible.

In the following and with reference to the further figures, a preferred embodiment of the nebulizer 1 and housing part 18 are described and shown, wherein primarily important aspects and differences will be described and the previous aspects, features and explanations apply preferably additionally or correspondingly even without repetition.

In the preferred embodiment, the nebulizer 1 or housing part 18 comprises a modified securing device 32 and/or a modified control part 40 according to the present invention.

FIG. 11 shows in a perspective view the modified securing device 32. FIG. 12 shows a view on the securing device 32 from above. FIG. 13 shows a section of the securing device 32 along line XIII-XIII of FIG. 12. FIG. 14 shows a side view of the securing device 32 in the same direction as the section of FIG. 13. FIG. 15 shows a section of the securing device 32 along line XV-XV of FIG. 12. FIG. 16 shows a side view of the securing device 32 in the same direction as the section of FIG. 15. FIG. 17 shows the securing device 32 in an exploded perspective view.

The securing device 32 comprises preferably the base 36 and a holding part 38.

Preferably, the holding part 38 and the base 36 are formed as separate parts which are joined or connected, in particular unseparably and/or before inserting the container 3 or with or after inserting the container 3.

With other words, the securing device 32 consists preferably of multiple parts, in particular metal parts.

Most preferably, the securing device 32 or one or more or all parts (here base 36 and holding part 38) are made of metal, in particular steel, most preferably spring steel.

The holding part 38 is preferably ring-like and/or essentially flat.

The securing device 32 or in particular holding part 38 comprises or supports preferably the holding elements 33. In the present embodiment, the holding elements 33 are preferably finger-like and/or tongue-like and/or extend essentially radially and/or inwardly. Preferably, the holding elements 33 are bent or curved upwards and, then, towards its free ends turn downwards and/or form essentially a half-circle in radial section. Thus, the free ends themselves form the holding surfaces or edges for the container 3, i.e. the free ends themselves define the boundary for any upwards movement of the container 3 within the securing device 32.

The orientation or position, such as upwards, downwards, upper, lower or the like mentioned above or used in the following, refers to the usual orientation of the nebulizer 1 or container 3 where the nebulizer 1 is at least essentially vertical and/or the outlet, nozzle 12 or mouthpiece 13 points upwards and/or the housing part 18 is at the bottom of the nebulizer 1 and/or the axial direction of the container 3 or its movement is vertical and/or the fluid outlet 24 or head of the container 3 is directed vertically upwards.

Preferably, the holding part 38 forms an upper part relative to the base 36.

Preferably, the holding element 33 and holding part 38 are made from one piece or unitary part, in particular by bending.

Preferably, the holding part 38 comprises or supports the fixing portions 37 for fixing the securing device 32, in particular in the housing part 18.

Preferably, the holding part 38 comprises or supports one or more locking portions 35 for holding the control part 40 in the non-activated state or delivery state and/or for preventing re-use or re-insertion of an already used container 3 or housing part 18 after detachment of the container 3 and housing part 18 from the nebulizer 1.

In the present embodiment, the securing device 32 or holding part 38 comprises preferably two locking portions 35, four fixing portions 37 and/or six holding elements 33.

Preferably, the base 36 comprises or supports the locking elements 34, the spring 20 with the piercing element 22 and/or connecting portions 43 for connecting the base 36 with the holding part 38.

The securing device 32 and in particular the base 36 and/or locking elements 34, form the transportation lock 39, preferably in cooperation with the holding elements 33 or any other axial stop (particularly in upwards direction) for container 3, as in the previous embodiment.

Preferably, the parts of the securing device 32, here the base 36 and upper or holding part 38, are connected unreleasable, in particular by crimping, snapping or welding.

In the present embodiment, the upper or holding part 38 and the base 36 are preferably spaced axially from each other and/or arranged essentially in different, axial offset planes.

Preferably, the base 36 and the holding part 38 are connected by means of the axially extending connecting portions 43. In particular, the connecting portions 43 form or comprise contact surfaces 48 (see FIG. 17) which rest against the ring-like part of the holding part 38 in the present embodiment.

In the present embodiment, the connecting portions 43 or contact surfaces comprise preferably gripping portions 44 which can snap or be crimped or bent to grip over an outer contour of the holding part 38. However, other constructional solutions are possible as well.

Alternatively and preferably, the two parts, in particular base 36 and upper or holding part 38, are connected by welding, riveting, hot calking or gluing and/or by means of screws (not shown). For these purposes, the holes shown for example in the contact surfaces 48 of the connecting portions 43 and in the holding part 38 can be used, e.g. for receiving screws, bolts, rivets or the like. Alternatively or additionally, the holes can be used for receiving alignment or fastening bolts or means in particular only temporarily during mounting.

FIG. 18 shows in a schematic section the nebulizer 1 with partly attached housing part 18, i.e. with (still) closed transportation lock 29 and with not (completely) closed housing of the nebulizer 1. FIG. 19 shows a partial enlargement of the encircled area of FIG. 18.

It has to be noted that the housing part 18 may be delivered together with the associated container 3 with closed transportation lock 29 separately from the nebulizer 1. However, it is also possible to pre-install the container 3 and housing part 18 in particular such that the container 3 is not yet fluidically opened or connected and that the housing is not yet completely closed.

When the transportation lock 29 is closed, the locking elements 34 are in the initial position locking the container 3 against axial movement, in the shown representations against downward movement and against opening or venting, in particular by piercing element 22.

In the present embodiment, the locking elements 34 do not hold or secure the container 3 by means of its free ends portions 34a, but preferably respectively by an arm portion 34c.

In particular, each locking elements 34 is angled such that the respective end portion 34a is supported or bent towards a lower region or base portion 34d adjacent to the base 36. In particular, the locking elements 34 form essentially a triangle in radial section. Thus, a very stable construction can be achieved, in particular such that very high locking forces can be realized when the transportation lock 29 is closed.

A further advantage of this construction is that the locking elements 34 do not open when the container 3 applies an axial force on the locking elements 34 or its arm portion 34c. The result is that the locking elements 34 and, thus, the transportation lock 29, open only by means of the control part 40 or any other actuation.

As shown in particular in FIG. 19, the holding part 38 and/or the holding elements 33 surround the container 3 even in the upper position, i.e. always. In particular, the container 3 extends with its radially extended lower and/or container base 21 or container edge 21 a below the main plane of the holding part 38.

Preferably, the fixing portions 37 extend towards the inner wall of housing part 18 and/or grip under shoulders 45, preferably extending radially inwardly and/or formed inside the nebulizer 1 or housing part 18.

Preferably, the securing device 32 or holding portion 38 can be pushed into the nebulizer 1 or housing part 18 such that the fixing portions 37 can pass, in particular with its free ends, the shoulders 45 and grip or snap under the shoulders 45 for holding or securing the securing device 32 by snap-fit or form-fit in place, in particular in the housing part 18.

The control part 40 comprises preferably at least one rib or arm 42 for opening the transportation lock 29, in particular for actuating, moving or pivoting an associated locking element 34 from the locking position shown in FIGS. 18 and 19 to the unlocking position shown in FIGS. 26 and 27.

FIG. 20 shows in a schematic perspective the cooperation of the control part 40 with the securing device 32. FIG. 20 shows only the container 3, the control part 40 and the securing device 32 without any other components of the nebulizer 1, in particular without the associated housing part 18. FIG. 20 shows the same state as FIGS. 18 and 19, i.e. the securing device 32 with closed transportation lock 29. FIG. 21 shows a partial enlargement of the encircled area A of FIG. 20 with regard to the cooperation of one locking portion 35 with the control part 40. FIG. 22 shows a partial enlargement of the encircled area B of FIG. 20 with regard to the cooperation of arms 42 of the control part 40 with one locking element 34.

In the non-activated state, with closed transportation lock 29 and/or with the control part 40 in its upper position, i.e. not yet moved downwardly and/or towards the securing device 32, the control part 40 is held by at least one locking portion 35, here by all locking portions 35, in particular in a snap-fit manner and/or by force-fit.

Preferably, two locking portions 35 are provided at opposite sides of the securing device 35 and/or holding part 38. The locking portions 35 extend axially and/or upwardly and/or are biased preferably inwardly.

FIG. 20 and in particular FIG. 21 show the preferred engagement of one locking portion 35 with or at the control part 40. In the present embodiment, the locking portion 35 comprises a recess or cut-out 35b, in particular two recesses or cut-outs 35b, near the free end of the locking portion 35.

The control part 40 comprises preferably at its ring portion 40a a protrusion 40b, in particular two protrusions 40b, engaging into the respective recesses or cut-outs 35b of the associated locking portion 35, to hold the control part 40 in the shown state. Preferably, the protrusions 40b are formed in an outer recess 40c of the control part 40 or ring portion 40a. Preferably, the locking portion 35 is guided by or through the preferably outer recess 40c when moving the control part 40 from the shown state or position downwardly to the moved position or towards the securing device 32.

The engagement of the protrusion(s) 40b into the cut-out(s) 35b forms the preferred snap-fit for holding the control part 40 in the present embodiment.

The locking portions 35 hold the control part 40 preferably in a defined rotational position, in particular by radial engagement into the outer recesses 40c and/or due to the cooperation with the protrusions 40b.

The control part 40 comprises preferably multiple ribs or arms 42 for actuating or opening the locking elements 34. Preferably, two arms 42 are associated to or actuate the same locking element 34.

Preferably, the securing device 32 or holding part 38 is received between the arms 42. For this purpose, the securing device 32 or holding part 38 comprises preferably a respectively indented outer contour, in particular indentions 46 as shown in particular in FIGS. 11, 12 and 17.

Preferably, the arms 42 extend between fixing portions 37 and/or locking portions 35 of the securing device 32 or holding part 38.

In the present embodiment, the securing device 32 comprises preferably three locking elements 34, in particular distributed regularly over the entire circumference, and the control part 40 comprises preferably six arms 42 cooperating with the locking elements 34.

The arms 42 extend preferably axially and/or towards or into the securing device 32 and/or parallel to each other.

In the delivery or initially state as shown in FIGS. 18 to 22, the at least one arm 42 preferably extends already to the actuation portion 34b, main plane or ring portion of the holding part 38 and/or further towards or into the securing device 32.

In the preferred embodiment, the arm 42 comprises a preferably inclined and/or actuation surface 41, here formed on its radial outside as shown in particular in FIG. 22.

In the preferred embodiment, the actuation portion 34b of the locking element 34 extends preferably transversely and/or at least essentially in circumferential direction, but preferably not axially as in the previous embodiment. Instead, preferably two actuation portions 34b extend transversally from a central portion 34e of the respective locking element 34. The central portion 34e is preferably narrower than the base portion 34d and extends from the base portion 34d to the actuation portions 34b of the respective locking element 34.

The arm portion 34c and/or base portion 34d are/is preferably wider than the central portion 34e.

The end portion 34a of the locking element 34 rests preferably against the edges of the base portion 34d on both sides of the central portion 34e and against the central portion 34e so that the end portion 34a cannot glide over the edges but is supported by form-fit engagement against depression resulting in that the arm portion 34c is supported in a very robust manner at its inner end bearing or holding the container 3 in its upward axial position with closed transportation lock 29.

In the present embodiment, the central portion 34e extends further above the actuation portions 34b and is angled or bent towards the arm portion 34c. In particular, a space is formed between each actuation portion 34b and the arm portion 34c so that the free end of arm 42 can pass through this space to securely actuate the locking element 34 at its respective actuation portion 34b.

The arm 42 or surface 41 of the arm 42 comprises preferably a notch 47 into which the locking element 34 or the associated actuation portion 34b can en-gage when the locking element 34 is actuated and/or when the transportation lock 29 is opened as shown in FIGS. 26 and 27.

When starting from the initial position, the control part 40 is moved downwardly, in particular by a component of the nebulizer 1, such as the inner part 17 or retaining part 39 or the like, wherein the initial holding or snapping force has to be overcome so that the snap-fit or form-fit or force-fit engagement of locking portions 35 with control part 40, in particular of cut-outs 35b with protrusions 40b, are released or overcome.

FIG. 23 shows in a similar view as FIG. 20 an intermediate position where the control part 40 has been moved already partially towards the securing device 32 and/or partially downwards towards the position where the transportation lock 29 is partly opened and/or the housing of the nebulizer 1 is not yet completely closed and/or the control part 40 is not yet in its lower end position within the housing part 18.

In the intermediate position, the arms 42 or surfaces 41 have already actuated or moved, in particular pivoted, the locking elements partially (outwards), in particular such that the container 3, container base 21 or container edge 21 a can (already) pass the locking elements 34 or arm portions 34c as shown in schematic section of FIG. 24.

During the downward movement, the locking elements 34 or actuation portions 34b glide over the arms 42 or surfaces 41 to progressively pivot or bend the locking elements 34 outwards or out of locking container 3, as indicated in FIG. 25.

FIG. 26 shows in a schematic sectional view the nebulizer 1 in the activated state, with completely closed housing, with completely closed or attached housing part 18 and/or with opened transportation lock 29, wherein the nebulizer 1 has already been tensioned and the holder 6 has already moved downwards and the container 3 has already been pierced or opened at the container base 21 by piercing element 22. FIG. 27 shows a partial enlargement of the encircled area of FIG. 26.

In this state, the locking elements 34 have reached the outer or non-locking positions. In this position, the locking elements 34 or its actuation portions 34b en-gage preferably into the respective notches 47 of the arms 42, in particular such that the arms 42 and, thus, the control part 40 are held in this moved or lower end position in a snap-fit or form-fit manner.

As the control part 40 is held or secured in the moved or lower end position, the transportation lock 29 is held open.

Further, the preferred snap-fit results in a defined axial position or fixation of the control part 40 relative to the securing device 32 or transportation lock 29, even if the housing part 18 is detached together with the securing device 32 and container 3 from the nebulizer 1, i.e. even if the retaining part 39 or any other component of the nebulizer 1 that abutted the control part 40 initially and/or caused the initial axial movement of the control part 40 is removed.

Therefore, the control part 40 is preferably held in snap-fit manner in the non-moved state with closed transportation lock 29 and/or in the axially moved state with opened transportation lock 29.

FIG. 28 shows in a schematic section when the housing part 18 is attached again to the nebulizer 1 wherein the housing part 18 and container 3 have already been used previously. In this case, the locking portions 35 have moved radially, in particular inwardly. In this context, it has to be noted that the locking portions 35 are preferably biased inwardly in this embodiment, and not outwardly as in the previous embodiment. Nevertheless, the locking portions 35 block (correct or complete) re-connection of the already used housing part 18, in particular complete closing of the housing of the nebulizer 1, preferably by abutment against inner part 17 or retaining part 39.

Preferably, the locking portions 35 are provided with steps so that the locking portions 35 tend to protrude radially inwardly from the connection with the holding part 38 towards the free ends of the locking elements 35. This assists the radial inward movement and locking against re-connection of an already used housing part 18 to a nebulizer 1. Further, the mentioned steps (preferably each locking element 35 preferably more than one radial step) assists also the movement of the control part 40 from its initial or upper position to its moved or lower position as the steps are preferably inclined with respect to the axial direction and the locking elements 35 glide on the control part 40 or vice versa when the control part 40 moves downwardly.

It is noted that the present embodiment comprising inwardly biased and/or locking portions 35 is different from the previous embodiment where the locking portions 35 are biased outwardly and/or move outwards for locking the housing part 18 against re-connection to a nebulizer 1.

In the present embodiment, the locking portions 35 extend preferably outside the inner part 17 when the nebulizer 1 is completely closed. This avoids any undesired interaction with drive spring 7 during the axial movement of the container 3 and holder 6.

As already mentioned, individual features, aspects and/or principles of the embodiments described may also be combined with one another as desired and may be used particularly in the nebulizers 1 according to FIGS. 1 and 10 but also in similar or different nebulizers.

Unlike freestanding equipment or the like the proposed nebulizer 1 is preferably designed to be portable and in particular is a mobile hand operated device.

The proposed solution may, however, be used not only in the nebulizers 1 specifically described here but also in other nebulizers or inhalers, e.g. powder inhalers or so-called metered dose inhalers.

Preferably, the fluid 2 is a liquid, as already mentioned, especially an aqueous pharmaceutical formulation or an ethanolic pharmaceutical formulation. However, it may also be some other pharmaceutical formulation, a suspension or the like.

According to an alternative embodiment the fluid 2 may also comprise particles or powder. In this case, instead of the expulsion nozzle 12, some other kind of supply device may be provided, especially an expulsion opening (not shown) or a supply channel (not shown) for supplying the fluid to or powder or the like into the mouthpiece 13. The optional air supply opening 15 then serves to supply ambient air preferably in parallel so as to general or allow an airflow with a sufficient volume for breathing in or inhaling through the mouthpiece 13.

If necessary the fluid 2 may also be atomized by means of a propellant gas.

Preferred ingredients and/or formulations of the preferably medicinal fluid 2 are listed in particular in WO 2009/115200 A1, preferably on pages 25 to 40, or in EP 2 614 848 A1, paragraphs 0040 to 0087, which are incorporated herewith by reference. In particular, these may be aqueous or non-aqueous solutions, mixtures, formulations containing ethanol or free from any solvent, or the like.

List of reference numerals

| | |
|---|---|
| 1 | nebulizer |
| 2 | fluid |
| 3 | container |
| 4 | bag |
| 5 | pressure generator |
| 6 | holder |
| 7 | drive spring |
| 8 | blocking element |
| 9 | conveying tube |
| 10 | non-return valve |
| 11 | pressure chamber |
| 12 | nozzle |
| 13 | mouthpiece |
| 14 | aerosol |
| 15 | air supply opening |
| 16 | upper housing part |
| 17 | inner part |
| 17a | upper part of the inner part |
| 17b | lower part of the inner part |
| 18 | housing part (lower part) |
| 19 | retaining element |
| 20 | spring |
| 21 | container base |
| 21a | container edge |
| 22 | piercing element |
| 23 | counter device |
| 24 | fluid outlet |
| 25 | first closure |
| 26 | second closure |
| 27 | closure part |
| 28 | flange |
| 29 | transportation lock |
| 30 | securing member |
| 31 | venting hole |
| 32 | securing device |
| 33 | holding element |
| 33a | end portion |
| 34 | locking element |
| 34a | end portion |
| 34b | actuation portion |
| 34c | arm portion |
| 34d | base portion |
| 34e | central portion |
| 35 | locking portion |
| 35a | protrusion |
| 35b | cut-out |
| 36 | base |
| 37 | fixing portion |
| 38 | holding part |
| 39 | retaining part |
| 40 | control part |
| 40a | ring portion |
| 40b | protrusion |
| 40c | outer recess |
| 41 | actuation surface |
| 42 | arm |
| 43 | connection portion |
| 44 | gripping portion |
| 45 | shoulder |
| 46 | indention |
| 47 | notch |
| 48 | contact surface |
| A | encircled area |
| B | encircled area |

What is claimed is:

1. A nebulizer (1) for a fluid (2), comprising:
a housing,
a container (3) containing the fluid (2) and being axially moveable within the housing in a activated state of the nebulizer (1) for conveying and/or nebulization of the fluid (2),
a securing device (32) forming a transportation lock (29) for holding the container (3) axially unmovable in a non-activated state of the nebulizer (1) with closed transportation lock (29), and
a control part (40) for opening the transportation lock (29), in particular when closing the housing and/or activating the nebulizer (1),
characterized in
that the control part (40) comprises at least one preferably axially extending rib or arm (42), wherein the control part (40) is moveable axially relative to or towards the securing device (32) for opening the transportation lock (29) by means of the at least one rib or arm (42), and/or
that the nebulizer (1) comprises a housing part which is comprised of (18) a container (3) containing the fluid (2),
a securing device (32) forming a transportation lock (29) for holding the container axially unmovable with closed transportation lock (29), and
a control part (40) for opening the transportation lock (29),
wherein the container (3) is inseparable from the housing part (18) by means of the securing device (32) and is axially moveable within the housing part (18) with opened transportation lock (29) for conveying and/or nebulization of the fluid (2),
characterized in
that the control part (40) comprises at least one preferably axially extending rib or arm (42), wherein the control part (40) is moveable relative to or towards the securing device (32) for opening the transportation lock (29) by means of the at least one rib or arm (42), and/or
that the securing device (32) comprises a ring-like holding part (38) with multiple holding elements (33) for holding the container (3) inseparably from the housing part (18), and comprises further a base (36) axially spaced from the holding part (38), wherein the base (36) comprises at least one locking element (34) for holding the container (3) axially unmovable with closed transportation lock (29).

2. The nebulizer according to claim 1, characterized in that the securing device (32) or transportation lock (29) comprises at least one locking element (34) for holding the container (3) axially unmovable with closed transportation lock (29), and that the rib or arm (42) comprises an actuation or inclined surface (41), wherein the locking element (34) is moveable or pivotable radially by means of the actuation or inclined surface (41) when moving the control part (40) axially relative to or towards the securing device (32) for opening the transportation lock (29).

3. The nebulizer according to claim 1, characterized in that the control part (40) comprises multiple ribs or arms (42) for opening the transportation lock (29), preferably wherein the securing device (32) or transportation lock (29) comprises multiple locking elements (34) for holding the container (3) axially unmovable with closed transportation lock (29).

4. The nebulizer according to claim 3, characterized in that the securing device (32) or at least a holding part (38) thereof is insertable or located in between the ribs or arms (42).

5. The nebulizer according to claim 1, characterized in that the securing device (32) comprises a base (36) preferably with locking elements (34) for holding the container (3) axially unmovable with closed transportation lock (29).

6. The nebulizer according to claim 5, characterized in that the securing device (32) comprises a holding part (38) with multiple holding elements (33) for holding the container (3) in the housing or a housing part (18) thereof inseparably.

7. The nebulizer according to claim 6, characterized in that the holding part (38) and the base (36) are axially offset and/or that the holding part (38) and the base (36) are separate parts.

8. The nebulizer according to claim 7, characterized in that the holding part (38) and the base (36) are connected with each other.

9. The nebulizer according to claim 8, characterized in that the holding part (38) and the base (36) are connected by means of connecting portions (43).

10. The nebulizer according to claim 6, characterized in that the holding elements (33) are finger-like and/or extend radially inwardly from the holding part (38).

11. The nebulizer according to claim 6, characterized in that the holding part (38) is ring-like and/or encircles the container (3).

12. The nebulizer according to claim 1, characterized in that the control part (40) is held in snap-fit manner, preferably by the securing device (32), in the non-moved state with closed transportation lock (29).

13. The nebulizer according to claim 1, characterized in that the control part (40) is held in snap-fit manner in the axially moved state with opened transportation lock (29).

14. The nebulizer according to claim 13, characterized in that at least one locking element (34) holds the control part (40) in snap-fit manner in the axially moved state with opened transportation lock (29).

15. A housing part (18) for a nebulizer (1) for dispensing a fluid (2), wherein the housing part (18) comprises:
a container (3) containing the fluid (2),
a securing device (32) forming a transportation lock (29) for holding the container axially unmovable with closed transportation lock (29), and
a control part (40) for opening the transportation lock (29),
wherein the container (3) is inseparable from the housing part (18) by means of the securing device (32) and is axially moveable within the housing part (18) with opened transportation lock (29) for conveying and/or nebulization of the fluid (2),
characterized in
that the control part (40) comprises at least one preferably axially extending rib or arm (42), wherein the control part (40) is moveable relative to or towards the securing device (32) for opening the transportation lock (29) by means of the at least one rib or arm (42), and/or
that the securing device (32) comprises a ring-like holding part (38) with multiple holding elements (33) for holding the container (3) inseparably from the housing part (18), and comprises further a base (36) axially spaced from the holding part (38), wherein the base (36) comprises at least one locking element (34) for holding the container (3) axially unmovable with closed transportation lock (29).

16. The housing part according to claim 15, characterized in that the securing device (32) or transportation lock (29) comprises at least one locking element (34) for holding the container (3) axially unmovable with closed transportation lock (29), and that the rib or arm (42) comprises an actuation or inclined surface (41), wherein the locking element (34) is moveable or pivotable radially by means of the actuation or inclined surface (41) when moving the control part (40) axially relative to or towards the securing device (32) for opening the transportation lock (29).

17. The housing part according to claim 15, characterized in that the control part (40) comprises multiple ribs or arms (42) for opening the transportation lock (29), preferably wherein the securing device (32) or transportation lock (29) comprises multiple locking elements (34) for holding the container (3) axially unmovable with closed transportation lock (29).

18. The housing part according to claim 17, characterized in that the securing device (32) or the holding part (38) is insertable or located in between the ribs or arms (42).

19. The housing part according to claim 15, characterized in that the holding part (38) and the base (36) are separate parts connected with each other, preferably by crimping, snapping or welding.

20. The housing part according to claim 15, characterized in that the holding elements (33) are finger-like and/or extend radially inwardly from the holding part (38).

21. The housing part according to claim 15, characterized in that the holding part (38) encircles the container (3).

22. The housing part according to claim 15, characterized in that the control part (40) is held in snap-fit manner, preferably by the securing device (32), in the non-activated state of the nebulizer (1) with closed transportation lock (29).
